(19) United States
(12) Patent Application Publication
(10) Pub. No.: US 2020/0038094 A1
(43) Pub. Date: Feb. 6, 2020

(54) TREATMENT SYSTEM

(71) Applicant: Olympus Corporation, Tokyo (JP)

(72) Inventor: Tsunetaka Akagane, Tokyo (JP)

(73) Assignee: Olympus Corporation, Tokyo (JP)

(21) Appl. No.: 16/600,405

(22) Filed: Oct. 11, 2019

Related U.S. Application Data
(63) Continuation of application No. PCT/JP2017/015296, filed on Apr. 14, 2017.

(51) Int. Cl. A61B 18/14

(52) U.S. Cl. CPC A61B 18/14; A61B 2018/1495; A61B 2018/1405

(57) ABSTRACT

An elongated treatment tool having a treatment portion disposed on a longitudinal axis thereof. The treatment portion includes a first treatment surface having a first electrode and a first insulative surface formed therein. The first electrode is disposed at a center in direction perpendicular to the longitudinal axis. The first insulative surface is disposed outwardly with respect to the first electrode. A second treatment surface having a second insulative surface and a second electrode formed therein. The second treatment surface is oriented in facing relationship with respect to the first treatment surface. When the first and second treatment surfaces are held in abutment against one another, the first insulative surface abuts against the second electrode and the second insulative surface and the second insulative surface abuts against the first electrode and the first insulative surface, thereby preventing a short circuit between the first electrode and the second electrode.

### CROSS-REFERENCE TO RELATED APPLICATION

This application is a continuation application of PCT Application No. PCT/JP 2017/015296 filed on Apr. 14, 2017, which is hereby incorporated by reference in its entirety.

### TECHNICAL FIELD

The disclosed technology relates generally to a treatment system, and more particularly, some embodiments relate to a treatment system for use with a treatment tool having electrodes and a heater.

### DESCRIPTION OF THE RELATED ART

US Patent Application Pub. No. 2001/0037109A1, for example, discloses a bipolar treatment tool. The treatment tool has a pair of treatment surfaces that face one another, each having an electrode and an insulative portion. When the pair of treatment surfaces are brought closest to one another, a gap is defined between the treatment surfaces. Therefore, when the treatment surfaces are brought closest to one another with no treatment target existing between the treatment surfaces, an electric current is prevented from flowing between the electrodes and hence a short circuit is prevented from developing therebetween.

For performing a treatment by passing a high-frequency current through a blood vessel, for example, to form a sealed region therein, it has been known that it is necessary to keep applying an appropriate pressure between the treatment surfaces at the position where the sealed region is to be formed from initial to terminal stages of the treatment. Furthermore, for performing a treatment by passing an electric current through a biological tissue, also known as, biotissue, for example, to coagulate the biotissue, in order to obtain a suitable coagulating performance, it is necessary to continue applying an appropriate pressure between the treatment surfaces at the position where the biotissue is to be coagulated from initial to terminal stages of the treatment.

### BRIEF SUMMARY OF EMBODIMENTS

The disclosed technology has been made in view of the foregoing. The disclosed technology to provide a treatment tool that is capable of continuously applying an appropriate gripping pressure between treatment surfaces to a treatment target when a high-frequency current is passed through the treatment target to treat the treatment target.

Accordingly, one aspect of the disclosed technology is directed to an elongated treatment tool having a treatment portion disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode.

Another aspect of the disclosed technology is directed to a treatment system comprises an energy source apparatus and an elongated treatment tool configured to be attached to the energy source apparatus to receive electrical energy. The elongated treatment tool having a treatment portion disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode.

A further aspect of the disclosed technology is directed to a treatment system comprises an energy source apparatus and an elongated treatment tool configured to be attached to the energy source apparatus to receive electrical energy. The elongated treatment tool includes a main body, a shaft, and a treatment portion all of which are attached to one another and are disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode while the electrode and the insulative surfaces are in flat contact with no gap.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view illustrating a treatment system according to first through eighth embodiments.

FIG. 2A is a schematic cross-sectional view, taken along line 2A-2A of FIG. 1, of a treatment portion of a treatment tool according to the first embodiment in the system illustrated in FIG. 1.

FIG. 2B is a schematic view illustrating a state in which a first treatment surface of a first treatment member and a second treatment surface of a second treatment member of the treatment portion illustrated in FIG. 2A abut against each other.

FIG. 2C is an enlarged view of the treatment portion at a position indicated by the numeral reference 2C in FIG. 2B.

FIG. 3A is a schematic view illustrating the first treatment surface of the first treatment member in the treatment portion illustrated in FIG. 1.

FIG. 3B is a schematic view illustrating the second treatment surface of the second treatment member in the treatment portion illustrated in FIG. 1.

FIG. 3C is a schematic view illustrating a first modification of the first treatment surface of the first treatment member in the treatment portion illustrated in FIG. 1.

FIG. 3D is a schematic view illustrating a first modification of the second treatment surface of the second treatment member in the treatment portion illustrated in FIG. 1.

FIG. 3E is a schematic view illustrating a second modification of the first treatment surface of the first treatment member in the treatment portion illustrated in FIG. 1.

FIG. 3F is a schematic view illustrating a second modification of the second treatment surface of the second treatment member in the treatment portion illustrated in FIG. 1.

FIG. 4A is a schematic cross-sectional view, taken along line 2A-2A of FIG. 1, of a treatment portion of a treatment tool according to the second embodiment in the system illustrated in FIG. 1.

FIG. 4B is a schematic view illustrating a state in which a first treatment surface of a first treatment member and a second treatment surface of a second treatment member of the treatment portion illustrated in FIG. 4A abut against each other.

FIG. 5A is a schematic cross-sectional view, taken along line 2A-2A of FIG. 1, of a treatment portion of a treatment tool according to the third embodiment in the system illustrated in FIG. 1.

FIG. 5B is a schematic view illustrating a state in which a first treatment surface of a first treatment member and a second treatment surface of a second treatment member of the treatment portion illustrated in FIG. 5A abut against each other.

FIG. 6A is a schematic cross-sectional view, taken along line 2A-2A of FIG. 1, of a treatment portion of a treatment tool according to the fourth embodiment in the system illustrated in FIG. 1.

FIG. 6B is a schematic view illustrating a state in which a first treatment surface of a first treatment member and a second treatment surface of a second treatment member of the treatment portion illustrated in FIG. 6A abut against each other.

FIG. 7A is a schematic cross-sectional view, taken along line 2A-2A of FIG. 1, of a treatment portion of a treatment tool according to the fifth embodiment in the system illustrated in FIG. 1.

FIG. 7B is a schematic view illustrating a state in which a first treatment surface of a first treatment member and a second treatment surface of a second treatment member of the treatment portion illustrated in FIG. 7A abut against each other.

FIG. 7C is an enlarged view of the treatment portion at a position indicated by the numeral reference 7C in FIG. 7B.

FIG. 8A is a schematic cross-sectional view, taken along line 2A-2A of FIG. 1, of a treatment portion of a treatment tool according to the sixth embodiment in the system illustrated in FIG. 1.

FIG. 8B is a schematic view illustrating a state in which a first treatment surface of a first treatment member and a second treatment surface of a second treatment member of the treatment portion illustrated in FIG. 8A abut against each other.

FIG. 9A is a schematic cross-sectional view, taken along line 2A-2A of FIG. 1, of a treatment portion of a treatment tool according to the seventh embodiment in the system illustrated in FIG. 1.

FIG. 9B is a schematic view illustrating a state in which a first treatment surface of a first treatment member and a second treatment surface of a second treatment member of the treatment portion illustrated in FIG. 9A abut against each other.

FIG. 10A is a schematic cross-sectional view along a longitudinal axis of a treatment portion of a treatment tool according to the eighth embodiment in the system illustrated in FIG. 1.

FIG. 10B is a schematic view illustrating a state in which a first treatment surface of a first treatment member and a second treatment surface of a second treatment member of the treatment portion illustrated in FIG. 10A abut against each other.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

It is an object of the disclosed technology to provide a treatment tool that is capable of continuously applying an appropriate gripping pressure between treatment surfaces to a treatment target from initial to terminal stages of the treatment. In addition, the disclosed technology is directed to make a structure capable of applying gripping pressure to any biotissue size, particularly very small sizes of biotissue, without causing

Electrical short circuit between electrodes. Thus, the electrode and the electrically insulative surfaces are in flat contact with no gap. A heating means such as a heater or an ultrasonic energy can be used to generate heat during a treatment.

Embodiments of the disclosed technology will be described hereinafter with reference to the drawings.

### First Embodiment

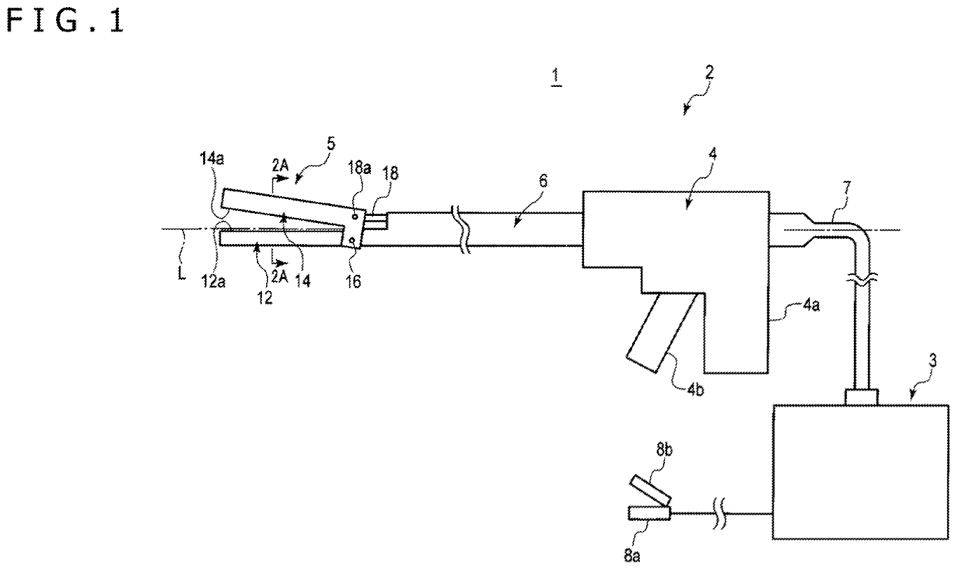

A first embodiment will be described hereinafter with reference to FIGS. 1 through 3B.

As illustrated in FIG. 1, a treatment system 1 has a treatment tool 2 (e.g., bipolar treatment tool) and a power supply 3.

The treatment tool 2 has a main body 4 and a treatment portion 5. A shaft 6 should preferably be disposed between the main body 4 and the treatment portion 5. The main body 4 is connected to a power supply 3 through a cable 7. A foot switch 8a, for example, is connected to the power supply 3. A switch, i.e., a hand switch, not illustrated, should preferably be disposed on the main body 4 together with or instead of the foot switch 8a.

The main body 4 has a fixed handle 4a integral with the main body 4 and a movable handle 4b movable toward and away from the fixed handle 4a. The treatment portion 5 has a first treatment member 12 and a second treatment member 14.

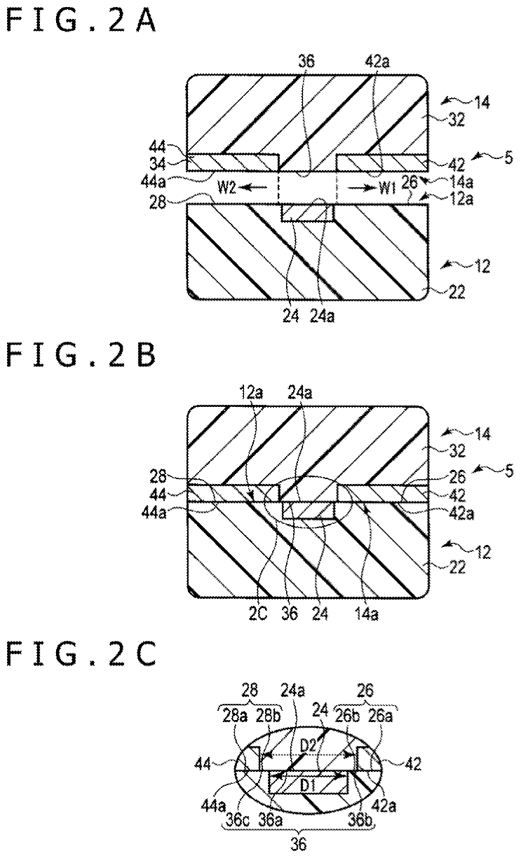

The main body 4 and the treatment portion 5 are disposed on an appropriate longitudinal axis L. The treatment portion 5 should preferably be longer in directions along the longitudinal axis L, i.e., longitudinal directions, than in widthwise directions W defined as directions perpendicular to the longitudinal axis L. In FIG. 2A, the widthwise directions W include a first direction indicated by the numeral reference W1 and a second direction indicated by the numeral reference W2. The first treatment member 12 and the second treatment member 14 are mutually angularly movably supported on a proximal end of the treatment portion 5 by a turn shaft 16. The turn shaft 16 should preferably extend perpendicularly to the longitudinal axis L and parallel to the widthwise directions W.

A drive shaft 18 is disposed between the main body 4 and the second treatment member 14 of the treatment portion 5. The drive shaft 18 is movable along the longitudinal axis L that represents a direction along which the treatment portion 5 extends from the main body 4. The drive shaft 18 is movable along the longitudinal axis L in ganged relation to the movable handle 4b as it moves. When the movable handle 4b is operated to move toward the fixed handle 4a of the main body 4, the drive shaft 18 is moved by a known mechanism to bring the second treatment member 14 that is coupled to a distal end 18a of the drive shaft 18 relatively toward the first treatment member 12. When the movable handle 4b is operated to move away from the fixed handle 4a, the drive shaft 18 is moved to bring the second treatment member 14 relatively away from the first treatment member 12.

The first treatment member 12 of the treatment portion 5 is fixed to the main body 4. When the movable handle 4b of the main body 4 is operated, for example, the second treatment member 14 moves with respect to the first treatment member 12. Specifically, a first jaw 22 of the first treatment member 12 is movable toward and away from a second jaw 32 of the second treatment member 14. Alternatively, the treatment portion 5 may be of such a structure that when the main body 4 is operated, both the first treatment member 12 and the second treatment member 14 move relatively to the main body 4. The treatment portion 5 that is of the former structure will be described hereinafter. Whether the treatment portion 5 is of the former structure or the latter structure, the second jaw 32 is relatively movable toward and away from the first jaw 22.

The power supply 3 is electrically connected to the treatment portion 5 through the main body 4. When a pedal 8b of the foot switch 8a, for example, is depressed by the user's foot, the power supply 3 supplies appropriate electric power to a first electrode 24 and a second electrode 34, to be described hereinafter, of the treatment portion 5, applying an appropriate voltage between the first electrode 24 and the second electrode 34. When the user releases the pedal 8b, the power supply 3 stops supplying electric power to the first electrode 24 and the second electrode 34.

As illustrated in FIGS. 1 through 3B, the first treatment member 12 of the treatment portion 5 has a first treatment surface, i.e., a gripper, 12a, and the second treatment member 14 has a second treatment surface, i.e., a gripper, 14a. The first treatment surface 12a of the first treatment member 12 faces the second treatment member 14. The second treatment surface 14a of the second treatment member 14 faces the first treatment member 12. The first treatment surface 12a and the second treatment surface 14a face each other. When the second treatment member 14 is angularly moved about the axis of the turn shaft 16 with respect to the first treatment member 12, the first treatment surface 12a and the second treatment surface 14a are moved toward and away from each other. The first treatment surface 12a and the second treatment surface 14a can grip a biotissue therebetween when they are moved toward each other. The first treatment surface 12a and the second treatment surface 14a can abut against each other when there is no biotissue present therebetween. Therefore, the treatment portion 5 of the treatment tool 2 according to the present embodiment can increase a gripping pressure on a thin treatment target such as a blood vessel or the like, compared with a treatment portion of a treatment tool that is of such a structure that when a first treatment surface and a second treatment surface are brought closely to each other, a spacer is disposed therebetween to keep the first treatment surface and the second treatment surface out of abutment against each other. The first treatment surface 12a and the second treatment surface 14a release the biotissue when they are separated from each other.

FIG. 2A illustrates a cross section taken along line 2A-2A of FIG. 1. Consequently, FIG. 2A illustrates a cross section of the treatment portion 5 perpendicular to the longitudinal axis L and substantially parallel to the widthwise directions W.

The first treatment member 12 has the first jaw 22, the first electrode 24, and the first treatment surface 12a disposed on the first jaw 22 first jaw 22 that moves toward or abuts against and moves away from the second treatment surface 14a. The first treatment surface 12a should preferably be formed as a planar surface. The second treatment member 14 has the second jaw 32, the second electrode 34, and second treatment surface 14a disposed on the second jaw 32. The second treatment surface 14a should preferably be formed as a planar surface.

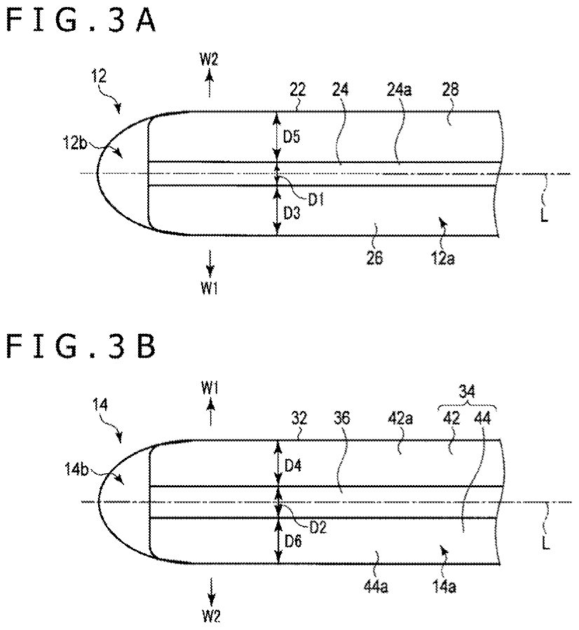

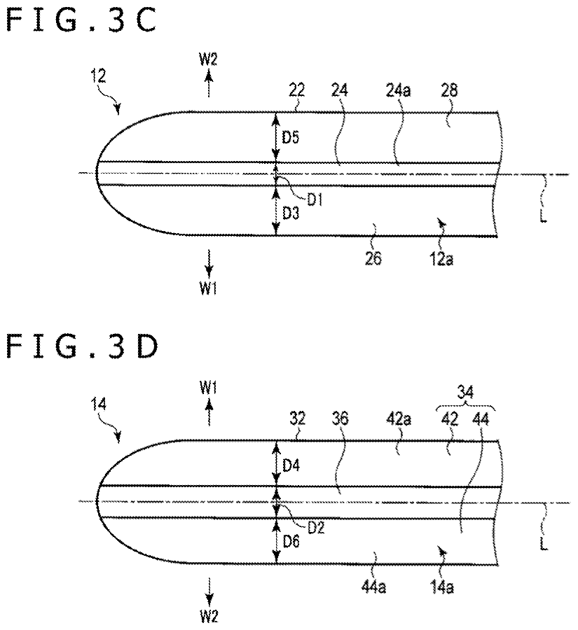

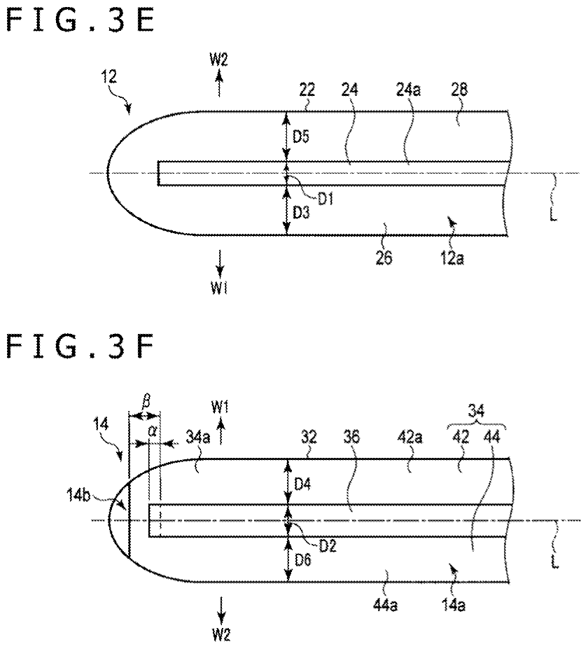

The first treatment surface 12a illustrated in FIG. 3A includes a distal-end surface 12b on a distal-end side thereof. The distal-end surface 12b should preferably be electrically insulative. The first treatment surface 12a and the distal-end surface 12b may lie or may not lie flush with each other. Similarly, the second treatment surface 14a illustrated in FIG. 3B includes a distal-end surface 14b on a distal-end side thereof. The distal-end surface 14b should preferably be electrically insulative. The second treatment surface 14a and the distal-end surface 14b may lie or may not lie flush with each other.

The first jaw 22 and the second jaw 32 extend along the longitudinal axis L. If the first jaw 22 and the second jaw 32 are made of a metal material that is electrically conductive, then the first jaw 22 and the second jaw 32 should preferably be covered with a material that is electrically insulative. The first jaw 22 and the second jaw 32 themselves may be made of a material that is electrically insulative which has appropriate rigidity. The first jaw 22 and the second jaw 32 should preferably have appropriate heat resistance. The first electrode 24 and the second electrode 34 are made of a material that is electrically conductive. The first electrode 24 and the second electrode 34 are used as different poles. Because of the electric insulation described hereinbefore, an unexpected electric current is prevented from flowing from the first electrode 24 to the first jaw 22. Similarly, an unintentional electric current is prevented from flowing from the second electrode 34 to the second jaw 32.

The first treatment surface 12a extends along the longitudinal axis L. The first treatment surface 12a has a first electrode surface, i.e., a surface for applying a gripping pressure, 24a defined by the first electrode 24, and planar portions, i.e., first insulative surfaces, 26 and 28 that are electrically insulative. The first planar portion 26 is disposed on the first direction W1 side of the first electrode surface 24a. The second planar portion 28 is disposed on the second direction W2 side of the first electrode surface 24a. According to the present embodiment, the first planar portion 26 and the second planar portion 28 that are integral with the first jaw 22 will be described by way of example. However, the first planar portion 26 and the second planar portion 28 may be separate from the first jaw 22.

The planar portions, i.e., surfaces for applying a gripping pressure, 26 and 28 are made of a material that, when heat caused by a high-frequency current is applied to a treatment target, e.g., a blood vessel or a biotissue, prevents the treatment target from sticking to the planar portions 26 and 28. The material of which the planar portions 26 and 28 are made should preferably be resistant to heat at approximately several hundred degrees, for example. The planar portions 26 and 28 of the first treatment surface 12a should preferably be made of fluororesin, for example, that is electrically insulative, as that material.

As illustrated in FIG. 3A, the first electrode 24 extends along the longitudinal axis L at the center of the first treatment surface 12a in the widthwise directions W. The planar portions 26 and 28 extend parallel to the longitudinal axis L at positions off the position along the longitudinal axis L at the center of the first treatment surface 12a in the widthwise directions W. Therefore, the first treatment surface 12a has the electrode 24 at the center thereof in the widthwise directions W and the planar portions 26 and 28 outside of the electrode 24 in the widthwise directions W.

The second treatment surface 14a extends along the longitudinal axis L. The second treatment surface 14a has planar portion, i.e., second insulative surface, 36 that is electrically insulative, and electrode surfaces, i.e., surfaces for applying a gripping pressure, 42a and 44a defined by a plurality of electrode members 42 and 44 into which the second electrode 34 is divided.

The planar portion, i.e., surfaces for applying a gripping pressure, 36 is made of a material that, when heat caused by a high-frequency current is applied to a treatment target, e.g., a blood vessel or a biotissue, prevents the treatment target from sticking to the planar portion 36. The material of which the planar portion 36is made should preferably be resistant to heat at approximately several hundred degrees, for example. The planar portion 36 of the second treatment surface 14a should preferably be made of fluororesin, for example, that is electrically insulative, as that material.

As illustrated in FIG. 3B, the planar portion, i.e., the second insulative surface, 36 extends along the longitudinal axis L at the center of the second treatment surface 14a in the widthwise directions W. The electrode surfaces 42a and 44a extend parallel to the longitudinal axis L at positions off the position along the longitudinal axis L at the center of the second treatment surface 14a in the widthwise directions W. Therefore, the second treatment surface 14a has the planar portion 36 at the center thereof in the widthwise directions W and the electrode surfaces 42a and 44a outside of the planar portion 36 in the widthwise directions W.

The first electrode member 42 is disposed on the first direction W1 side of the planar portion 36 formed by the second jaw 32. The second electrode member 44 is disposed on the second direction W2 side of the planar portion 36 formed by the second jaw 32. The electrode members 42 and 44 of the second electrode 34 are of the same pole and kept at the same potential. The potential is defined as voltage.

The electrode surface 24a of the first treatment surface 12a faces the planar portion 36 of the second treatment surface 14a. The planar portion 26 of the first treatment surface 12a faces the electrode surface 42a of the second treatment surface 14a. The planar portion 28 of the first treatment surface 12a faces the electrode surface 44a of the second treatment surface 14a.

As illustrated in FIG. 2C, the first planar portion 26 has a first abutment surface, i.e., an electrode abutment surface, 26a for abutting against the first electrode surface 42a, and a second abutment surface, i.e., an insulation abutment surface, 26b for abutting against the planar portion 36. The first abutment surface 26a and the second abutment surface 26b are contiguous to each other. The second planar portion 28 has a third abutment surface, i.e., an electrode abutment surface, 28a for abutting against the second electrode surface 44a, and a fourth abutment surface, i.e., an insulation abutment surface, 28b for abutting against the planar portion 36. The third abutment surface 28a and the second abutment surface 28b are contiguous to each other.

The planar portion 36 of the second treatment surface 14a has a first abutment surface, i.e., an electrode abutment surface, 36a for abutting against the electrode surface 24a, a second abutment surface, i.e., an insulation abutment surface, 36b that is contiguous to the first abutment surface 36a, for abutting against the first planar portion 26, and a third abutment surface, i.e., an insulation abutment surface, 36c that is contiguous to the second abutment surface 36a, for abutting against the second planar portion 28.

The boundary between the electrode surface 24a and the second abutment surface 26b of the planar portion 26 and the boundary between the electrode surface 24a and the fourth abutment surface 28b of the planar portion 28 should preferably lie flush with each other. The boundary between the electrode surface 42a and the second abutment surface 36b of the planar portion 36 and the boundary between the electrode surface 44a and the third abutment surface 36c of the planar portion 36 should preferably lie flush with each other.

Although not illustrated, spaces may be defined between the electrode surface 24a and the second abutment surface 26b of the planar portion 26 and between the electrode surface 24a and the fourth abutment surface 28b of the planar portion 28. In addition, spaces may be defined between the electrode surface 42a and the second abutment surface 36b of the planar portion 36 and between the electrode surface 44a and the third abutment surface 36c of the planar portion 36.

According to the present embodiment, for the sake of brevity, it is assumed that the first treatment surface 12a and the second treatment surface 14a have the same width in the widthwise directions W. With the first treatment surface 12a and the second treatment surface 14a in abutment against each other, a widthwise dimension D1 of the electrode surface 24a of the first treatment surface 12a is smaller than a widthwise dimension D2 of the planar portion 36 of the second treatment surface 14a. With the first treatment surface 12a and the second treatment surface 14a in abutment against each other, a widthwise dimension D3 of the planar portion 26 of the first treatment surface 12a is larger than a widthwise dimension D4 of the electrode surface 42a of the second treatment surface 14a. Similarly, with the first treatment surface 12a and the second treatment surface 14a in abutment against each other, a widthwise dimension D5 of the planar portion 28 of the first treatment surface 12a is larger than a widthwise dimension D6 of the electrode surface 44a of the second treatment surface 14a. Therefore, the length of the planar portions 26 and 28 along the widthwise directions W is larger than the length of the second electrode 34 along the widthwise directions W. Moreover, the length of the planar portion 36 along the widthwise directions W is larger than the length of the first electrode 24 along the widthwise directions W.

Next, operation of the treatment tool 2 according to the present embodiment will be described hereinafter.

The user of the treatment tool 2 moves the movable handle 4b of the main body 4 toward the fixed handle 4a until the second treatment surface 14a abuts against the first treatment surface 12a.

The first abutment surface 26a of the first planar portion 26 of the first treatment surface 12a abuts against the electrode surface 42a of the electrode member 42 of the second treatment surface 14a in a planar fashion. At this time, the first abutment surface 26a of the first planar portion 26 of the first treatment surface 12a abuts against the electrode surface 42a of the electrode member 42 of the second treatment surface 14a in either of the directions along the longitudinal axis L and the widthwise directions W perpendicular to the longitudinal axis L.

The third abutment surface 28a of the second planar portion 28 of the first treatment surface 12a abuts against the electrode surface 44a of the electrode member 44 of the second treatment surface 14a in a planar fashion. At this time, the third abutment surface 28a of the second planar portion 28 of the first treatment surface 12a abuts against the electrode surface 44a of the electrode member 44 of the second treatment surface 14a in either of the directions along the longitudinal axis L and the widthwise directions W perpendicular to the longitudinal axis L.

Therefore, the planar portions, i.e., first areas, 26 and 28 have the respective abutment surfaces 26a and 28a abutting respectively against the electrode members 42 and 44 of the second electrode 34 in a planar fashion.

The first abutment surface 36a of the planar portion, i.e., second area, 36 of the second treatment surface 14a abuts against the electrode surface 24a of the first treatment surface 12a in a planar fashion. At this time, the first abutment surface 36a of the planar portion 36 of the second treatment surface 14a abuts against the electrode surface 24a of the first treatment surface 12a in either of the directions along the longitudinal axis L and the widthwise directions W perpendicular to the longitudinal axis L.

Of the planar portion 26 of the first treatment surface 12a, the second abutment surface 26b that is closer to the center in the widthwise directions W abuts against the second abutment surface 36b, positioned toward the first direction W1 of the widthwise directions W, of the planar portion 36 of the second treatment surface 14a. Of the planar portion 28 of the first treatment surface 12a, the fourth abutment surface 28b that is closer to the center in the widthwise directions W abuts against the third abutment surface 36c, positioned toward the second direction W2 of the widthwise directions W, of the planar portion 36 of the second treatment surface 14a. In view of wobbling movements, etc. of the second treatment member 14 with respect to the first treatment member 12, the width, i.e., abutting area, between the second abutment surface 26b and the second abutment surface 36b and the width, i.e., abutting area, between the fourth abutment surface 28b and the third abutment surface 36c are set to appropriate values.

Consequently, the first treatment surface 12a has the planar portions, i.e., surfaces for applying a gripping pressure, 26 and 28 that include the abutment surfaces 26a and 28a for abutting against the second electrode 34, i.e., the electrode surfaces 42a and 44a in a planar fashion. Furthermore, the second treatment surface 14a has the planar portion, i.e., a surface for applying a gripping pressure, 36 for abutting against the planar portions 26 and 28, the planar portion 36 including the abutment surface 36a for abutting against the first electrode 24, i.e., the electrode surface 24a in a planar fashion.

Therefore, even when the first treatment surface 12a and the second treatment surface 14a are held in abutment against each other, the first electrode 24 and the second electrode 34 are disposed in positions spaced from each other. Specifically, the first electrode 24 and the second electrode 34 are spaced from each other in at least either the directions along the longitudinal axis L or the widthwise directions W perpendicular to the longitudinal axis L. Consequently, even when the pedal 8b of the foot switch 8a is pressed to pass a high-frequency current between the first electrode 24 and the second electrode 34, a short circuit is prevented from developing between the first electrode 24 and the second electrode 34.

When the first treatment surface 12a and the second treatment surface 14a of the treatment portion 5 of the treatment tool 2 according to the present embodiment are held in abutment against each other, no gap is present in opening and closing directions, perpendicular to the longitudinal axis L and the widthwise directions W, of the first treatment surface 12a and the second treatment surface 14a. Therefore, even if a tissue gripped between the first treatment surface 12a and the second treatment surface 14a is a thin tissue, a gripping pressure is transmitted to the tissue.

Moreover, no spacer is present between the first treatment surface 12a and the second treatment surface 14a. Consequently, a gripping pressure acting on a biotissue as a treatment target between the first treatment surface 12a and the second treatment surface 14a is restrained from changing largely along the widthwise directions W. In addition, a biotissue as a treatment target is easily gripped in a larger area between the first treatment surface 12a and the second treatment surface 14a.

A treatment, i.e., an electrifying treatment, for passing a high-frequency current through a blood vessel, not illustrated, to form a sealed region therein, using the treatment portion 5 of the treatment tool 2 according to the present embodiment will be described by way of example hereinafter. It should be noted that it is within the scope of the disclosed technology that, an ultrasonic energy can be used to generate heat for the treatment.

A blood vessel as a treatment target is gripped between the first treatment surface 12a and the second treatment surface 14a. The blood vessel is gripped while in contact with both the first treatment surface 12a and the second treatment surface 14a. At this time, the blood vessel extends out of the treatment portion 5 along the widthwise directions W, for example.

The blood vessel is gripped between the electrode surface 24a and the planar portion 36, between the abutment surface 26a and the electrode surface 42a, and between the abutment surface 28a and the electrode surface 44a. Therefore, the blood vessel is held in contact with both the electrode 24 of the first treatment surface 12a and the electrode 34 of the second treatment surface 14a, i.e., the electrode members 42 and 44, while kept under a gripping pressure. Respective paths through the blood vessel between the first electrode 24 and the electrode member 42 of the second electrode 34 and between the first electrode 24 and the electrode member 44 of the second electrode 34 are made short.

When the user presses the pedal 8b of the foot switch 8a, electric power is supplied from the power supply 3 through the main body 4 of the treatment tool 2 to the first electrode 24 and the second electrode 34, applying a voltage between the first electrode 24 and the second electrode 34. A high-frequency current thus flows through the blood vessel gripped between the first electrode 24 and the second electrode 34. In other words, the high-frequency current is applied to a portion of the blood vessel as the treatment target where a sealed region is to be formed. At this time, heat caused by the high-frequency current is applied to not only positions near the electrode surfaces 42a and 44a of the electrode members 42 and 44, but also the blood vessel between the electrode surfaces 42a and 44a of the electrode members 42 and 44, between the electrode surface 24a and the electrode surfaces 42a and 44a of the electrode members 42 and 44. Therefore, the length of the blood vessel along a width D1 in the widthwise directions W of at least the electrode surface 24a can be affected by the heat caused by the high-frequency current. Alternatively, an ultrasonic energy can be used to generate heat for the treatment. The blood vessel between the first electrode 24 and the second electrode 34, i.e., the electrode members 42 and 44 thereof, is progressively dehydrated and dried, and hence made thin by the electrifying treatment. At this time, the distance between the first treatment surface 12a and the second treatment surface 14a, i.e., the distance in the opening and closing directions, is reduced as the blood vessel becomes thinner.

It is known that obtaining a good sealing performance using the treatment tool 2 for performing an electrifying treatment on a blood vessel to form a sealed region therein depends upon not only the state of the blood vessel, but also the gripping pressure applied to the blood vessel.

The sealing performance for blood vessels is required to withstand an appropriate blood pressure of several hundreds mmHg, for example. Since the sealing performance is possibly subject to variations, it is preferable to set the sealing performance of the treatment tool 2 such that it can withstand a high blood pressure of 1000 mmHg, for example.

The first treatment surface 12a and the second treatment surface 14a of the treatment portion 5 of the treatment tool 2 according to the present embodiment are configured between themselves into a state able to abut against each other. Therefore, as the treatment to seal a blood vessel progresses and the blood vessel becomes progressively thinner, the gripping pressure on the blood vessel rises. When the treatment, i.e., the electrifying treatment, to seal the blood vessel is about to be finished, a maximum gripping pressure is applied to the blood vessel. Consequently, appropriate gripping pressures are continuously applied to the blood vessel from the initial to terminal stages of the treatment. Therefore, the blood vessel is well sealed using the spacerless and gapless treatment tool 2 in which the first treatment surface 12a and the second treatment surface 14a abut against each other. In other words, an appropriate sealed region is formed in the blood vessel.

When heat is applied to a blood vessel to form a sealed region therein, the blood vessel may shrink toward the center thereof in the widthwise directions W. As the blood vessel shrinks, a force is applied to open the treatment surfaces 12a and 14a relatively to each other.

The gripping pressure between the first treatment surface 12a and the second treatment surface 14a can be increased as the electrifying treatment of the treatment target is in progress. Therefore, the blood vessel is prevented as much as possible from shrinking toward the center in the widthwise directions W. Therefore, the gripping pressure is kept applied to the blood vessel between the first treatment surface 12a and the second treatment surface 14a from the initial to terminal stages of the treatment. The gripping pressure between the first treatment surface 12a and the second treatment surface 14a prevents the biotissue as the treatment target from shrinking, i.e., from gathering toward the center in the widthwise directions W, as the treatment is in progress.

The example in which the treatment is performed by supplying electric power from the high-frequency power supply 3a to the electrodes 24 and 34 to form a sealed region in a blood vessel has been described hereinbefore. A similar treatment is carried out to coagulate a treatment target of a biotissue.

As described hereinbefore, the treatment tool 2 according to the present embodiment deserves to be commented as follows:

If there is no biotissue present between the first treatment surface 12a and the second treatment surface 14a, then there is no gap between the first treatment surface 12a and the second treatment surface 14a. Therefore, when a biotissue is gripped between the first treatment surface 12a and the second treatment surface 14a, the treatment surfaces 12a and 14a apply a gripping pressure to the treatment target at all times regardless of whether the biotissue is thin or is made thin by an electrifying treatment. Therefore, an electric current can be passed between the first electrode 24 and the second electrode 34 while the biotissue is being strongly compressed therebetween.

At this time, since there is no gap present between the first treatment surface 12a and the second treatment surface 14a, the first treatment surface 12a and the second treatment surface 14a can grip the biotissue that is thin or is made thin by an electrifying treatment, in a wider area thereof. Consequently, forces are less likely to concentrate on one location of the biotissue, preventing the biotissue from being incised unexpectedly during the treatment.

For forming a sealed region in a blood vessel, for example, the first treatment surface 12a and the second treatment surface 14a grip the blood vessel in a wider area thereof. Even if the blood vessel is thin or the blood vessel becomes progressively thinner as the treatment progresses, an appropriate gripping pressure can be applied to the blood vessel continuously from the initial to terminal stages of the electrifying treatment. Therefore, the sealed state of the sealed region of the blood vessel is stabilized. Moreover, the blood pressure resistance of the blood vessel, i.e., the difficulty with which the blood flows through the blood vessel, is increased by the sealed region.

Therefore, the treatment tool 2 according to the present embodiment is capable of continuously applying an appropriate gripping pressure between the treatment surfaces 12a and 14a to a treatment target such as a blood vessel, a biotissue, or the like that becomes thinner as an electrifying treatment progresses. Accordingly, the treatment portion 5 of the treatment tool 2 according to the present embodiment is able to increase the gripping pressure on a thin treatment target such as a blood vessel or the like, compared with a treatment portion of a treatment tool having such a structure that a spacer is disposed between a first treatment surface and a second treatment surface when they come close to each other, preventing the first treatment surface and the second treatment surface from abutting against each other.

According to the present embodiment, the example in which the first treatment surface 12a has the single electrode surface 24a and the two planar portions, i.e., insulative surfaces, 26 and 28 and the second treatment surface 14a has the two electrode surfaces 42a and 44a and the single planar portion, i.e., insulative surface, 36 has been described hereinbefore. However, the first treatment surface 12a may have two electrode surfaces and single planar portion, i.e., insulative surface, and the second treatment surface 14a may have a single electrode surface and two single planar portion, i.e., insulative surfaces. Therefore, the first treatment surface 12a and the second treatment surface 14a may have a single electrode member or a plurality of electrode members.

In the example illustrated in FIG. 3A, the distal-end surface 12b that is electrically insulative is disposed on the distal-end side of the first treatment surface 12a. Therefore, the distal end of the electrode surface 24a is positioned closer to the proximal end of the first treatment member 12 than the distal end thereof. In the example illustrated in FIG. 3B, the distal-end surface 14b is disposed on the distal-end side of the second treatment surface 14a. Therefore, the distal end of the planar portion 36 that faces the electrode surface 24a is positioned closer to the proximal end of the second treatment member 14 than the distal end thereof.

FIG. 3C illustrates a first modification of the first treatment surface 12a of the first treatment member 12. FIG. 3D illustrates a first modification of the second treatment surface 14a of the second treatment member 14.

As illustrated in FIG. 3C, the distal-end side of the first treatment surface 12a is free of the distal-end surface 12b (see FIG. 3A) that is electrically insulative. The distal end of the electrode surface 24a is aligned with the distal end of the first treatment member 12. In case the treatment surface 12a of the treatment member 12 is in the state illustrated in FIG. 3C, the distal-end side of the second treatment surface 14a is free of the distal-end surface 14b (see FIG. 3B) that is electrically insulative. The planar portion 36 that faces the electrode surface 24a is in an area including the distal end of the second treatment member 14 so as to abut against the electrode surface 24a illustrated in FIG. 3C. In this case, the electrode surfaces 42a and 44a have distal ends disposed in the area which may including the distal end of the second treatment member 14, or positioned closer to the proximal end of the second treatment member 14 than the distal end thereof.

FIG. 3E illustrates a second modification of the first treatment surface 12a of the first treatment member 12. FIG. 3F illustrates a second modification of the second treatment surface 14a of the second treatment member 14.

As illustrated in FIG. 3E, the distal-end side of the first treatment surface 12a is free of the distal-end surface 12b (see FIG. 3A) that is electrically insulative. The distal end of the electrode surface 24a is positioned closer to the proximal end of the first treatment member 12 than the distal end thereof. In case the treatment surface 12a of the first treatment member 12 is in the state illustrated in FIG. 3E, the distal-end portion of the planar portion 36 of the second treatment surface 14a protrudes a distance α (>0) from the distal end of the electrode surface 24a of the first treatment surface 12a as illustrated in FIG. 3F. The electrode 34 that includes the electrode surfaces 42a and 44a has an electrode surface 34a that is contiguous in an area between the distal end of the planar portion 36 and the distal-end surface 14b that is electrically insulative. Therefore, the electrode 34 is substantially U-shaped on the second treatment surface 14a. A broken line near the distal end of the planar portion 36 illustrated in FIG. 3F represents a position that becomes closest to the distal end of the electrode surface 24a of the first treatment surface 12a when the first treatment surface 12a and the second treatment surface 14a are relatively closed. Therefore, when the first treatment surface 12a and the second treatment surface 14a are relatively closed, the distal end of the electrode surface 24a abuts against or is close to the planar portion 36 that is electrically insulative. The distal-end surface 14b that is electrically insulative is disposed on the distal-end side of the distal end of the electrode surface 34a, i.e., the electrode surfaces 42a and 44a. The distal end of the electrode surface 34a, i.e., the electrode surfaces 42a and 44a protrudes a distance β (>α>0) from the broken line near the distal end of the planar portion 36 illustrated in FIG. 3F. Therefore, the distal end of the second treatment member 14 is electrically insulative.

The treatment performance can be varied by the structure in the vicinity of the distal-end portion of the first treatment surface 12a side of the first treatment member 12 and in the vicinity of the distal-end portion of the second treatment surface 14a side of the second treatment member 14.

An electrifying treatment using a treatment portion 5 according to a first modification illustrated in FIGS. 3C and 3D is able to form a sealed region in a blood vessel or coagulate a biotissue by adjusting the width D1, etc. of the electrode surface 24a and/or adjusting the output of electric power in the similar manner as described hereinbefore.

Here, the electrode surface 24a of the electrode 24 exists particularly in the distal end of the first treatment surface 12a along the longitudinal axis L. Furthermore, the electrode surfaces 42a and 44a of the electrode 34 exist in the vicinity of the distal end of the second treatment surface 14a along the longitudinal axis L. Therefore, the areas of the first treatment surface 12a and the second treatment surface 14a in the vicinity of their distal ends along the longitudinal axis L form a substantially straight coagulated region or sealed surface in a biotissue when an electric current is passed through the biotissue between the electrode surface 24a and the electrode surfaces 42a and 44a. The treatment portion 5 according to the first modification can coagulate the biotissue over substantially the entire lengths of first treatment surface 12a and the second treatment surface 14a including their distal ends along the longitudinal axis L.

An electrifying treatment using a treatment portion 5 according to a second modification illustrated in FIGS. 3E and 3F is able to form a sealed region in a blood vessel or coagulate a biotissue in the similar manner as described hereinbefore.

In the vicinity of the distal end of the first treatment surface 12a, illustrated in FIG. 3E, of the treatment portion 5 according to the second modification, the distal end of the electrode surface 24a of the electrode 24 is disposed at a position spaced from the distal end of the first treatment surface 12a toward the proximal end thereof along the longitudinal axis L. In other words, the electrode surface 24a of the electrode 24 does not exist at the position of the most distal end of the first treatment surface 12a. On the second treatment surface 14a illustrated in FIG. 3F, the electrode surface 34a of the electrode 34 is substantially U-shaped in surrounding relation to the planar portion 36. The areas of the first treatment surface 12a and the second treatment surface 14a in the vicinity of their distal ends along the longitudinal axis L form a substantially U-shaped coagulated region or sealed surface in a biotissue when an electric current is passed through the biotissue between the electrode surface 24a and the electrode surface 34a. As described hereinbefore, the distal end of the electrode surface 24a of the electrode 24 is disposed at a position spaced from the distal end of the first treatment surface 12a toward the proximal end thereof along the longitudinal axis L. Furthermore, the electrode surface 34a has a distal end disposed at a position spaced from the distal end of the second treatment surface 14a toward the proximal end thereof along the longitudinal axis L with the electrically insulative distal-end surface 14b of the second treatment surface 14a being interposed therebetween. Therefore, no coagulated region or sealed surface of the biotissue is formed at the position of the most distal ends of the first treatment surface 12a and the second treatment surface 14a along the longitudinal axis L.

As described hereinbefore, the portion of the first treatment surface 12a of the first treatment member 12 in the vicinity of its distal-end portion and the portion of the second treatment surface 14a of the second treatment member 14 in the vicinity of its distal-end portion are not limited to the structures illustrated in FIGS. 3A and 3B. The portion of the first treatment surface 12a in the vicinity of its distal-end portion and the portion of the second treatment surface 14a in the vicinity of its distal-end portion may be, for example, of the structures illustrated in FIGS. 3C and 3D according to the first modification or the structures illustrated in FIGS. 3E and 3F according to the second modification. The portion of the first treatment surface 12a in the vicinity of its distal-end portion and the portion of the second treatment surface 14a in the vicinity of its distal-end portion may be of other various shapes.

### Second Embodiment

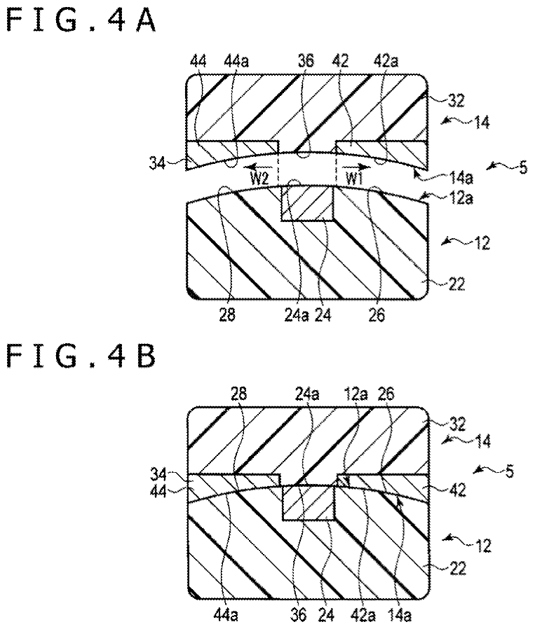

A second embodiment will be described hereinafter with reference to FIGS. 4A and 4B. The second embodiment is a modification of the first embodiment. Those parts of the second embodiment that are identical or have identical functions to those parts described in the first embodiment are denoted if at all possible by identical numeral references, and will not be described in detail hereinafter. This also holds true for a third through eighth embodiment to be described hereinafter. The structures according to the first through eighth embodiments can appropriately be combined with each other.

In the first embodiment described hereinbefore, the first treatment surface 12a and the second treatment surface 14a are illustrated as flat. However, the first treatment surface 12a and the second treatment surface 14a may be curved, as illustrated in FIGS. 4A and 4B.

In the example illustrated in FIGS. 4A and 4B, the first treatment surface 12a is shaped as a projected surface and the second treatment surface 14a is shaped as a recessed surface. Although not illustrated, the first treatment surface 12a may be shaped as a recessed surface and the second treatment surface 14a may be shaped as a projected surface.

### Third Embodiment

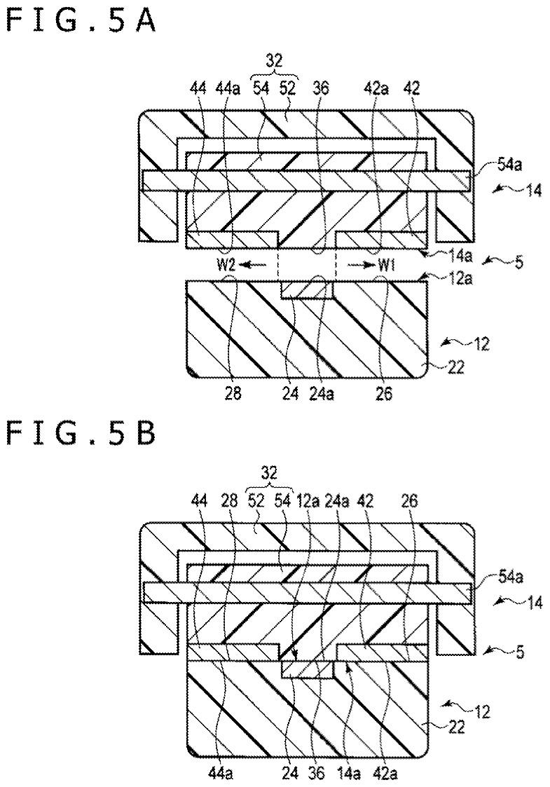

Next, a third embodiment of the disclosed technology will be described hereinafter with reference to FIGS. 5A and 5B.

When the first treatment surface 12a and the second treatment surface 14a of the treatment portion 5 according to the first embodiment described hereinbefore are brought closely to each other, the proximal-end sides thereof along the longitudinal axis L become close to each other faster than the distal-end sides thereof. Therefore, when a biotissue is to be gripped, it may possibly undergo different gripping pressures in the directions along the longitudinal axis L. When a biotissue is to be gripped, it can undergo a uniformized gripping pressure in the widthwise directions W.

As illustrated in FIGS. 5A and 5B, the second treatment member 14 includes a jaw body 52 and a turn member 54 angularly movably supported on the jaw body 52 by a turn shaft 54a. The turn shaft 16 and the distal end 18a of the drive shaft 18 illustrated in FIG. 1 are disposed on the jaw body 52. The jaw body 52 has its outer peripheral surface covered with a material that is electrically insulative. The turn member 54 has the second treatment surface 14a.

When an operation is made to bring the movable handle 4b toward the fixed handle 4a of the main body, the drive shaft 18 is moved to cause the jaw body 52 coupled to the distal end 18a of the drive shaft 18 to move relatively closer to the first treatment member 12. At this time, in ganged relation to the movement of the jaw body 52, the turn member 54 is angularly moved about the turn shaft 54a relatively closer to the first treatment member 12. This mechanism makes the first treatment surface 12a and the second treatment surface 14a of the turn member 54 parallel or substantially parallel to each other.

With the second treatment member 14 thus arranged, the gripping pressure for gripping a biotissue can be uniformized in not only the widthwise directions W, but also the directions along the longitudinal axis L, compared with the example described in the first embodiment described hereinbefore. Consequently, using the treatment portion 5 of the treatment tool 2 according to the present embodiment, it is easier to perform a treatment for coagulating a biotissue well or a treatment for sealing a blood vessel well than the example described in the first embodiment.

In the present embodiment, as illustrated in FIGS. 5A and 5B, the example in which the second jaw 32 of the second treatment member 14 is made up of the jaw body 52 and the turn member 54 has been described hereinbefore. The first jaw 22 of the first treatment member 12 may also be similarly constructed.

### Fourth Embodiment

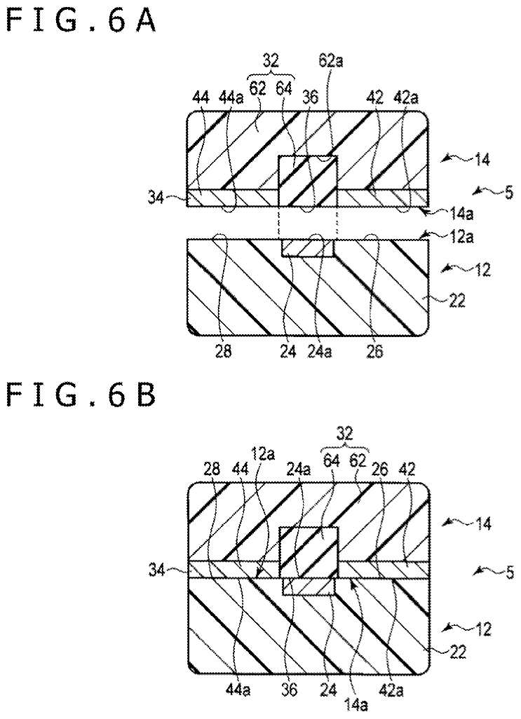

Next, a fourth embodiment of the disclosed technology will be described hereinafter with reference to FIGS. 6A and 6B.

The second jaw 32 of the treatment portion 5 according to the first embodiment described hereinbefore has been described as being unitary.

As illustrated in FIGS. 6A and 6B, the second jaw 32 of the second treatment member 14 has a jaw body 62 and a pad 64 disposed on the jaw body 62. The second jaw 32 may thus be made up of a plurality of members. The jaw body 62 has a recess 62a defined therein that extends along the longitudinal axis L. The pad 64 is fixedly disposed in the recess 62a in the jaw body 62. The pad 64 extends in the jaw body 62 along the longitudinal axis L at the second treatment surface 14a.

The jaw body 62 has at least its outer peripheral surface, i.e., an externally exposed portion thereof that is electrically insulative. The pad 64 is electrically insulative. The pad 64 is heat-resistant. The pad 64 should preferably be made of a soft material compared with the jaw body 62. The planar portion, i.e., the insulative surface, 36 is defined by the pad 64.

The planar portion, i.e., the insulative surface, 36 of the pad 64 at the second treatment surface 14a is used in the similar manner as the planar portion 36 described in the first embodiment.

The first planar portion 26 and the second planar portion 28 of the first treatment surface 12a may be made of the same material as the pad 64.

### Fifth Embodiment

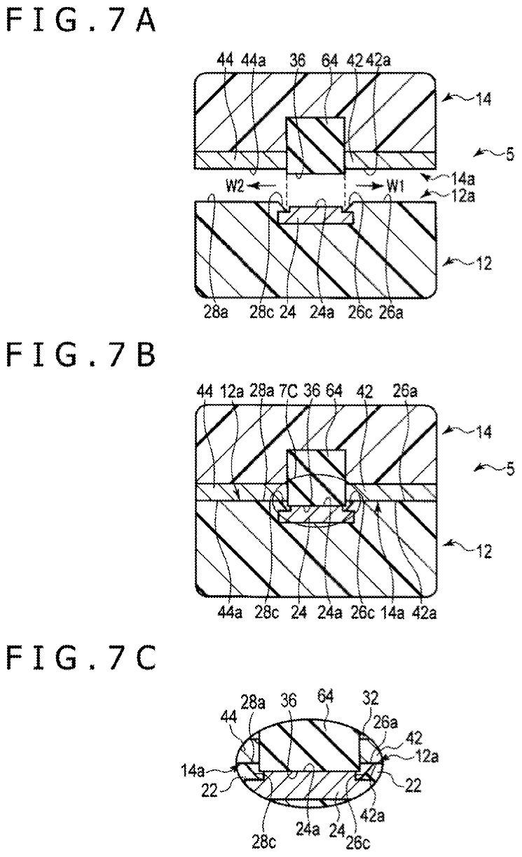

Next, a fifth embodiment of the disclosed technology will be described hereinafter with reference to FIGS. 7A through 7C.

In the first embodiment described hereinbefore, the example in which no gap exists between the first treatment surface 12a and the second treatment surface 14a has been described hereinbefore. According to an example illustrated in FIGS. 7A through 7C, the first treatment surface 12a and the second treatment surface 14a have respective steps and a gap is defined in a region between the first treatment surface 12a and the second treatment surface 14a.

The planar portions, i.e., the insulative surfaces, 26 and 28 of the first treatment surface 12a protrude toward the second treatment surface 14a with respect to the electrode surface 24a of the electrode 24 that is disposed adjacent thereto on the central side in the widthwise directions W. Specifically, the abutment surface, i.e., the electrode abutment surface, 26a of the planar portion 26 protrudes toward the second treatment surface 14a with respect to the electrode surface 24a of the electrode 24. The planar portion 26 has a slanted surface 26c lying between the abutment surface, i.e., a surface for applying a gripping pressure, 26a and the electrode surface 24a and contiguous to the abutment surface 26a. The slanted surface 26c makes the abutment surface 26a of the planar portion 26 protrude toward the second treatment surface 14a with respect to the electrode surface 24a. Similarly, the abutment surface, i.e., the electrode abutment surface, 28a of the planar portion 28 protrudes toward the second treatment surface 14a with respect to the electrode surface 24a of the electrode 24. The planar portion 28 has a slanted surface 28c lying between the abutment surface, i.e., a surface for applying a gripping pressure, 28a and the electrode surface 24a and contiguous to the abutment surface 28a. The slanted surface 28c makes the abutment surface 28a of the planar portion 28 protrude toward the second treatment surface 14a with respect to the electrode surface 24a. According to the present embodiment, therefore, the first treatment surface 12a is shaped as a non-flat surface.

The planar portion, i.e., the insulative surface, 36 of the second treatment surface 14a protrudes toward the first treatment surface 12a with respect to the electrode surface 42a that is adjacent to the planar portion 36 in the first direction W1 of the widthwise directions W and the electrode surface 44a that is adjacent to the planar portion 36 in the second direction W2 of the widthwise directions W. The planar portion, i.e., a surface for applying a gripping pressure, 36 is defined by the pad 64.

The planar portion 36 protrudes toward the electrode surface 24a of the first treatment surface 12a progressively from the outer sides toward the center in the widthwise directions W. According to the present embodiment, therefore, the second treatment surface 14a is shaped as a non-flat surface. The planar portion 36 can abut against the electrode surface 24a of the first treatment surface 12a.

When the planar portion 36 is held in abutment against the electrode surface 24a of the first treatment surface 12a, the abutment surface 26a of the planar portion 26 and the electrode surface 42a of the electrode member 42 abut against each other and the abutment surface 28a of the planar portion 28 and the electrode surface 44a of the electrode member 44 abut against each other.

Next, operation of the treatment tool 2 according to the present embodiment will be described hereinafter.

When the second treatment surface 14a is brought into abutment against the first treatment surface 12a, the electrode surface 24a and the planar portion 36 abut against each other in a planar fashion, the abutment surface 26a and the electrode surface 42a abut against each other in a planar fashion, and the abutment surface 28a and the electrode surface 44a abut against each other in a planar fashion. Furthermore, when the second treatment surface 14a is brought into abutment against the first treatment surface 12a, gaps are defined between the slanted surface 26c and the planar portion 36 as well as the electrode surface 42a and between the slanted surface 28c and the planar portion 36 as well as the electrode surface 44a.

Therefore, when a high-frequency current flows between the first electrode 24 and the second electrode 34, i.e., the electrode members 42 and 44, a short circuit is prevented from developing between the first electrode 24 and the second electrode 34. Of the electrode surface 42a, the area closer to the center in the widthwise directions W faces the slanted surface 26c along the directions in which the first treatment surface 12a and the second treatment surface 14a are opened and closed. Of the electrode surface 44a, the area closer to the center in the widthwise directions W faces the slanted surface 28c along the directions in which the first treatment surface 12a and the second treatment surface 14a are opened and closed. The electrode surface 24a and the electrode surface 42a are close to each other, and the electrode surface 24a and the electrode surface 44a are close to each other.

When the second treatment surface 14a is brought into abutment against the first treatment surface 12a, the electrode surface 24a at the center in the widthwise directions W and the planar portion 36 abut against each other, the abutment surface 26a spaced from the center in the first direction W1 and the electrode surface 42a abut against each other, and the abutment surface 28a spaced from the center in the second direction W2 and the electrode surface 44a abut against each other. In particular, the abutment surface 26a and the electrode surface 42a, and the abutment surface 28a and the electrode surface 44a abut against each other in a planar fashion. Therefore, since the abutment surface 26a and the electrode surface 42a, and the abutment surface 28a and the electrode surface 44a, of the first treatment surface 12a and the second treatment surface 14a of the treatment portion 5 of the treatment tool 2 according to the present embodiment, abut against each other in a planar fashion, there are no gaps therebetween along the directions in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

A treatment, i.e., an electrifying treatment, for passing a high-frequency current through a blood vessel, not illustrated, to form a sealed region therein, using the treatment portion 5 of the treatment tool 2 according to the present embodiment will be described by way of example hereinafter.

In the similar manner as described in the first embodiment, a blood vessel as a treatment target is gripped between the first treatment surface 12a and the second treatment surface 14a. The blood vessel is gripped while in contact with both the first treatment surface 12a and the second treatment surface 14a.

There are gaps defined between the slanted surface 26c and the planar portion 36 as well as the electrode surface 42a, and between the slanted surface 28c and the planar portion 36 as well as the electrode surface 44a. A blood vessel is gripped between the electrode surface 24a and the planar portion 36, between the abutment surface 26a and the electrode surface 42a, and between the abutment surface 28a and the electrode surface 44a. Therefore, the blood vessel is held in contact with both the electrode 24 of the first treatment surface 12a and the electrode 34, i.e., the electrode members 42 and 44, of the second treatment surface 14a while under the gripping pressure. When a high-frequency current is applied to a portion of the blood vessel as the treatment target where a sealed region is to be formed, the blood vessel between the first electrode 24 and the second electrode 34, i.e., the electrode members 42 and 44, is progressively dehydrated and dried, and becomes thinner. The distance between the first treatment surface 12a and the second treatment surface 14a becomes smaller as the blood vessel is thinner.

Consequently, the treatment portion 5 of the treatment tool 2 according to the present embodiment applies a maximum gripping pressure when it is about to finish the treatment to seal the blood vessel. Consequently, appropriate gripping pressures are continuously applied to the blood vessel between the electrode surface 24a and the planar portion 36, between the abutment surface 26a and the electrode surface 42a, and between the abutment surface 28a and the electrode surface 44a from the initial to terminal stages of the treatment. Therefore, the blood vessel is well sealed using the spacerless and gapless treatment tool 2 in which the first treatment surface 12a and the second treatment surface 14a abut against each other in a planar fashion. In other words, an appropriate sealed region is formed in the blood vessel.

The example in which the treatment is performed by pressing the first switch 8a to supply electric power from the high-frequency power supply 3a to the electrodes 24 and 34 to form a sealed region in a blood vessel has been described hereinbefore. A similar treatment is carried out to coagulate a treatment target of a biotissue.

Therefore, the treatment tool 2 according to the present embodiment is capable of continuously applying an appropriate gripping pressure between the treatment surfaces 12a and 14a to a treatment target such as a blood vessel, a biotissue, or the like that becomes thinner as an electrifying treatment progresses.

In the example illustrated in FIGS. 7A through 7C, the area of contact between the electrode surface 24a of the electrode 24 of the first treatment surface 12a and the planar portion 36 of the second treatment surface 14a is large in not only the directions along the longitudinal axis L, but also the widthwise directions W, and they contact each other in a planar fashion. The area of contact between the electrode surface 24a of the electrode 24 of the first treatment surface 12a and the planar portion 36 of the second treatment surface 14a may be smaller in the widthwise directions W. In this case, there may be an instance in which the second planar portion, i.e., the insulative surface, 36, or the abutment surface 36a thereof, does not abut against the electrode surface 24a of the first electrode 24 in a planar fashion.

### Sixth Embodiment

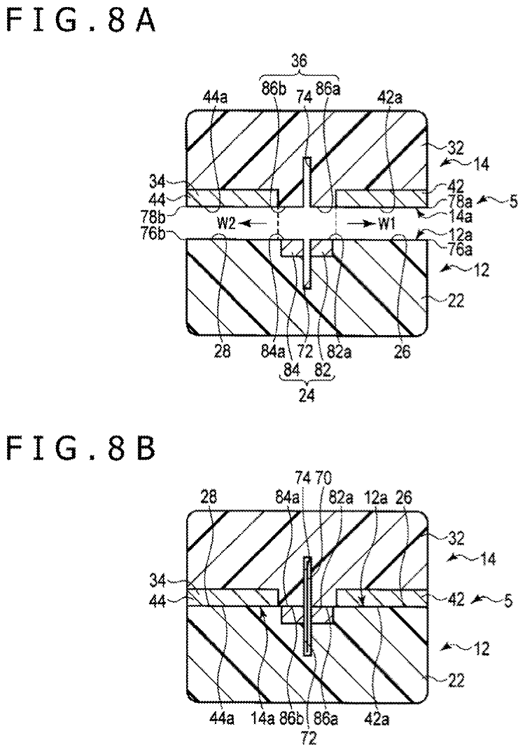

Next, a sixth embodiment of the disclosed technology will be described hereinafter with reference to FIGS. 8A and 8B.

In the first embodiment described hereinbefore, the example in which no gaps at all are present between the first treatment surface 12a and the second treatment surface 14a has been described hereinbefore.

As illustrated in FIGS. 8A and 8B, a cutter 70 is guided into and out of slits 72 and 74 that are defined in the first treatment surface 12a and the second treatment surface 14a and extend along the longitudinal axis L. According to the present embodiment, a discrete region, i.e., the slit 72, is defined in the first treatment surface 12a between an end 76a and another end 76b thereof in the widthwise directions W. A discrete region, i.e., the slit 74, is defined in the second treatment surface 14a between an end 78a and another end 78b thereof in the widthwise directions W. The slits 72 and 74 make up a single space that is contiguous in the directions along the longitudinal axis L when the first treatment surface 12a and the second treatment surface 14a abut against each other.

The slit 72 is illustrated as being defined in the center in the widthwise directions W of the first treatment surface 12a, and the slit 74 is illustrated as being defined in the center in the widthwise directions W of the second treatment surface 14a. In FIGS. 8A and 8B, the slit 72 divides the electrode 24 into electrode members 82 and 84 that are of the same pole and kept at the same potential. The first treatment surface 12a is made up of the first planar portion 26, the second planar portion 28, an electrode surface 82a of the electrode member 82 of the electrode 24, and an electrode surface 84a of the electrode member 84 of the electrode 24. In FIGS. 8A and 8B, the slit 74 divides the planar portion 36 into planar portions, i.e., insulative surfaces, 86a and 86b. The second treatment surface 14a is made up of the first electrode surface 42a, the second electrode surface 44a, and the planar portions 86a and 86b.

A substantially U-shaped peripheral surface that is defined by the slits 72 and 74 illustrated in FIG. 8A is basically kept out of contact with a biotissue. Therefore, the substantially U-shaped surface that is defined by the slits 72 and 74 does not use as a treatment surface for sealing a blood vessel or coagulating a biotissue.

Therefore, when the first treatment surface 12a and the second treatment surface 14a of the treatment portion 5 of the treatment tool 2 according to the present embodiment abut against each other, there are no gaps present along the directions, perpendicular to the longitudinal axis L and the widthwise directions W, in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

Though the slits 72 and 74 are defined in the first treatment surface 12a and the second treatment surface 14a of the treatment portion 5 of the treatment tool 2 according to the present embodiment, the first treatment surface 12a and the second treatment surface 14a are configured between themselves into a state able to abut against each other. Therefore, as the treatment to seal a blood vessel progresses and the blood vessel becomes progressively thinner, the gripping pressure on the blood vessel rises. When the treatment, i.e., the electrifying treatment, to seal the blood vessel is about to be finished, a maximum gripping pressure is applied to the blood vessel. Consequently, appropriate gripping pressures are continuously applied to the blood vessel from the initial to terminal stages of the treatment. Therefore, the blood vessel is well sealed even though the slits 72 and 74 are defined in the first treatment surface 12a and the second treatment surface 14a.

After the treatment for sealing the blood vessel is finished, the cutter 70 is guided through the slits 72 and 74 from the proximal-end side toward the distal-end side along the longitudinal axis L. The cutter 70 then appropriately cuts the blood vessel that has been dried by the treatment.

In the example illustrated in FIGS. 8A and 8B, the slit 72 is illustrated as being defined in the center in the widthwise directions W of the first treatment surface 12a and the slit 74 is illustrated as being defined in the center in the widthwise directions W of the second treatment surface 14a. Otherwise, the slit 72 may be defined in the first treatment surface 12a at a position shifted from the center in the widthwise directions W of the first treatment surface 12a and the slit 74 may be defined in the second treatment surface 14a at a position shifted from the center in the widthwise directions W of the second treatment surface 14a.

### Seventh Embodiment

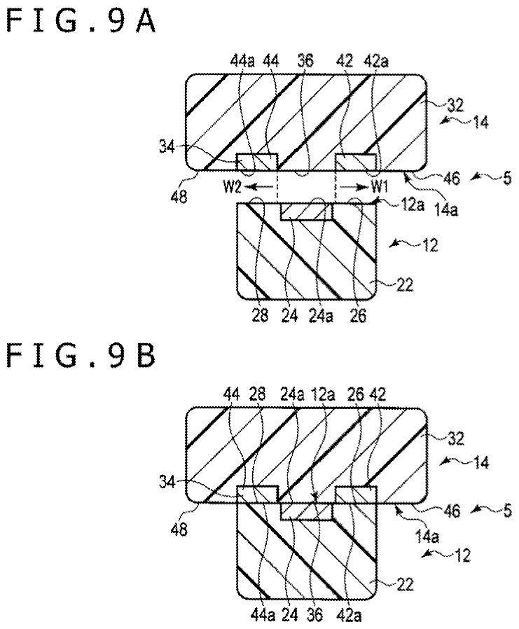

Next, a seventh embodiment of the disclosed technology will be described hereinafter with reference to FIGS. 9A and 9B.

In the first embodiment described hereinbefore, the example in which the widths of the first treatment surface 12a and the second treatment surface 14a in the widthwise directions W are the same as each other has been described. In addition, the example in which the electrode surfaces are disposed at positions on the second treatment surface 14a that are spaced from the center in the widthwise directions W has been described.

As illustrated in FIGS. 9A and 9B, the widths of the first treatment surface 12a and the second treatment surface 14a in the widthwise directions W are different from each other. Furthermore, surfaces that are electrically insulative, rather than electrode surfaces, are disposed at positions on the second treatment surface 14a that are spaced from the center in the widthwise directions W.

In the similar manner as described in the first embodiment, the first treatment surface 12a is made up of the first electrode surface 24a of the first electrode 24, the first planar portion 26, and the second planar portion 28. The second treatment surface 14a is made up of the planar portion 36, the electrode surface 42a of the first electrode member 42, the electrode surface 44a of the second electrode member 44, the planar portion, i.e., the insulative surface, 46 extending from the electrode surface 42a in the first direction W1 along the widthwise directions W, and the planar portion, i.e., the insulative surface, 48 extending from the electrode surface 44a in the second direction W2 along the widthwise directions W. When the first treatment surface 12a and the second treatment surface 14a abut against each other, the electrode surface 42a of the first electrode member 42 and the electrode surface 44a of the second electrode member 44 should preferably not be exposed outwardly. In other words, the both ends of the first treatment surface 12a should preferably be positioned outwardly of the electrode surface 42a of the first electrode member 42 and the electrode surface 44a of the second electrode member 44.

As described hereinbefore, the first treatment surface 12a and the second treatment surface 14a may not necessarily be of the same width.

Even if the planar portions, i.e., the insulative surfaces, 46 and 48 contact or support a biotissue while a high-frequency current is flowing between the electrodes 24 and 34, the planar portions 46 and 48 do not apply an energy directly to the biotissue.

### Eighth Embodiment

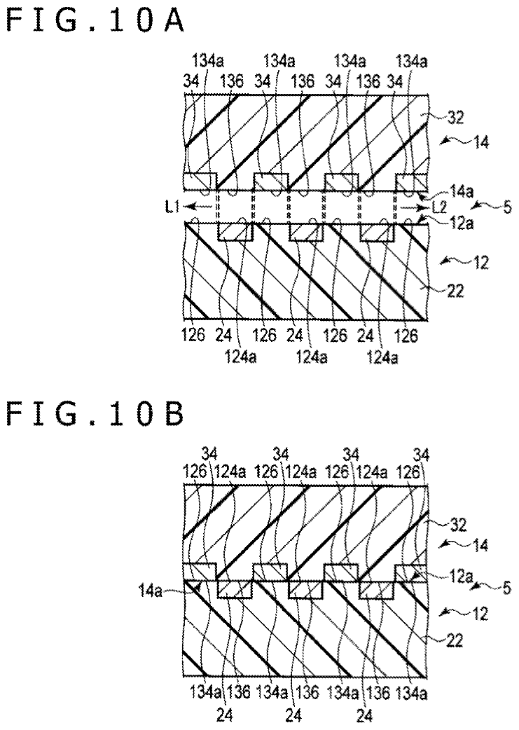

Next, an eighth embodiment of the disclosed technology will be described hereinafter with reference to FIGS. 10A and 10B.

In the first through seventh embodiments, the cross sections of the first treatment member 12 and the second treatment member 14, taken perpendicularly to the longitudinal axis L, have been illustrated, and it has been described that when the first treatment surface 12a and the second treatment surface 14a abut against each other, the electrode 24 of the first treatment member 12 and the electrode 34 of the second treatment member 14 are spaced from each other.

FIGS. 10A and 10B illustrate portions of cross sections along the longitudinal axis L of the first treatment member 12 and the second treatment member 14. The manner in which when the first treatment surface 12a and the second treatment surface 14a abut against each other, the electrode 24 of the first treatment member 12 and the electrode 34 of the second treatment member 14 are spaced from each other will briefly be described hereinafter. The directions along the longitudinal axis L include a first direction, i.e., a distal-end direction, denoted by the numeral reference L1 in FIG. 10A, and a second direction, i.e., a proximal-end direction, denoted by the numeral reference L2 in FIG. 10A.

The first treatment surface 12a has a plurality of electrode surfaces 124a defined by the first electrode 24 and planar portions, i.e., insulative surfaces, 126 defined by the first jaw 22 and disposed between the electrode surfaces 124a. The second treatment surface 14a has a plurality of electrode surfaces 134a defined by the second electrode 34 and planar portions, i.e., insulative surfaces, 136 defined by the second jaw 32 and disposed between the electrode surfaces 134a.

The first treatment surface 12a and the second treatment surface 14a abut against each other in the directions along the longitudinal axis L. When the second treatment surface 14a is brought into abutment against the first treatment surface 12a, the electrode surfaces 124a of the first treatment surface 12a abut against only the first planar portions 136 of the second treatment surface 14a, and do not abut against the electrode surfaces 134a. Similarly, the electrode surfaces 134a of the second treatment surface 14a abut against only the planar portions 126 of the first treatment surface 12a, and do not abut against the electrode surfaces 124a.

Therefore, the pedal 8b of the foot switch 8a is depressed to pass a high-frequency current between the first electrode 124 and the second electrode 134, a short circuit is prevented from developing between the first electrode 124 and the second electrode 134.

When the first treatment surface 12a and the second treatment surface 14a of the treatment portion 5 of the treatment tool 2 according to the first embodiment are brought into abutment against each other, there are no gaps present along the directions in which the first treatment surface 12a and the second treatment surface 14a along the longitudinal axis L are opened and closed. Consequently, even if a tissue gripped between the first treatment surface 12a and the second treatment surface 14a is a thin tissue, the gripping pressure is transmitted to the tissue. When a biotissue is gripped between the first treatment surface 12a and the second treatment surface 14a, even if the biotissue is thin or made thin by a treatment, the paired treatment surfaces 12a and 14a apply a pressure to the biotissue at all times. Therefore, an electric current can be passed between the first electrode 24 and the second electrode 34 while the biotissue is being strongly compressed.

Therefore, the treatment tool 2 according to the present embodiment is capable of continuously applying an appropriate gripping pressure between the treatment surfaces 12a and 14a to a treatment target such as a blood vessel, a biotissue, or the like that becomes thinner as an electrifying treatment progresses.

Providing the treatment portion 5 having the structure according to the third embodiment illustrated in FIGS. 5A and 5B is applied to the second treatment member 14, for example, the gripping pressure for gripping a biotissue can be uniformized in not only the first direction L1 and the second direction L2, but also the directions along the longitudinal axis L.

The example in which the first treatment member 12 has the electrode 24 and the second treatment member 14 has the electrode 34 has been described with respect to the treatment portions 5 of the treatment tools 2 according to the first through eighth embodiments. A heater may be attached to the reverse side of at least one of the electrodes 24 and 34. The electrode itself to which the heater is attached is used as a heat transfer member. When the heater is energized, the temperature of the electrode surface may be increased to an appropriate temperature ranging from 100° C. to several hundreds degrees Celsius. In this case, a biotissue as a treatment target can be coagulated and a blood vessel as a treatment target can be sealed by an action of the heat from the heater and an action of a high-frequency current.

The certain embodiments have hereinbefore been described in specific detail with reference to the drawings. The disclosed technology is not limited to the embodiments described hereinbefore, but covers all embodiments that may be carried out without departing from the scope of the invention.

In sum, one aspect of the disclosed technology is directed to an elongated treatment tool having a treatment portion disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode.

The second electrode is made up of a plurality of separate electrode members having the same pole and kept at the same electrical potential or voltage. The first insulative surface has an electrode abutment surface for abutting against the second electrode and an insulation abutment surface for abutting the second insulative surface. Either the first treatment surface or the second treatment surface is formed as a projected surface. The treatment tool further comprises a heating means configured to be used to treat a biotissue during the treatment. The heating means is a heater or the heating means is an ultrasonic energy to generate heat for the treatment. The first electrode includes a first surface. The second electrode includes a second surface. A length of the first insulative surface along the widthwise directions is larger than a length of the second surface along the widthwise directions and a length of the second insulative surface along the widthwise directions is larger than a length of the first surface along the widthwise directions. Each of the first insulative surface and the second insulative surface is made of fluororesin. The second insulative surface is defined by a pad that is electrically insulative. The first electrode includes a first surface and the second electrode includes a second surface. The first treatment surface is a flat surface by the first insulative surface and the first surface. The second treatment surface is a flat surface by the second insulative surface and the second surface. The first electrode includes a first surface, the second electrode includes a second surface. At least one of a boundary between the first insulative surface and the first surface on the first treatment surface and a boundary between the second insulative surface and the second surface on the second treatment surface is formed as a flush surface. The first treatment surface extends along the longitudinal axis. The first treatment surface and the second treatment surface have slits defined respectively therein that extend along the longitudinal axis. A cutter is capable of being brought into and out of the slits along the longitudinal axis.

Another aspect of the disclosed technology is directed to a treatment system comprises an energy source apparatus and an elongated treatment tool configured to be attached to the energy source apparatus to receive electrical energy. The elongated treatment tool having a treatment portion disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode.

A further aspect of the disclosed technology is directed to a treatment system comprises an energy source apparatus and an elongated treatment tool configured to be attached to the energy source apparatus to receive electrical energy. The elongated treatment tool includes a main body, a shaft, and a treatment portion all of which are attached to one another and are disposed on a longitudinal axis thereof. The treatment portion includes a first treatment surface having a first electrode and a first insulative surface formed therein. The first electrode extends along the longitudinal axis and is disposed at a center in widthwise directions perpendicular to the longitudinal axis and the first insulative surface is disposed outwardly with respect to the first electrode in the widthwise directions. A second treatment surface having a second insulative surface and a second electrode formed therein. The second insulative surface extends along the longitudinal axis and is disposed at the center in the widthwise directions perpendicular to the longitudinal axis and the second electrode is disposed outwardly with respect to the second insulative surface in the widthwise directions. The second treatment surface is oriented in facing relationship with respect to the first treatment surface and angularly movable about a turn shaft parallel to the first treatment surface and into abutment against the first treatment surface. When the first treatment surface and the second treatment surface are held in abutment against one another, the first insulative surface abuts against the second electrode and the second insulative surface and the second insulative surface abuts against the first electrode and the first insulative surface, thereby preventing a short circuit between the first electrode and the second electrode while the electrode and the insulative surfaces are in flat contact with no gap.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An elongated treatment tool having a treatment portion disposed on a longitudinal axis thereof,
the treatment portion including:
a first treatment surface having a first electrode and a first insulative surface formed therein, the first electrode extends along the longitudinal axis and being disposed at a center in widthwise directions perpendicular to the longitudinal axis and the first insulative surface disposed outwardly with respect to the first electrode in the widthwise directions; and
a second treatment surface having a second insulative surface and a second electrode formed therein, the second insulative surface extends along the longitudinal axis and being disposed at the center in the widthwise directions perpendicular to the longitudinal axis and the second electrode disposed outwardly with respect to the second insulative surface in the widthwise directions, the second treatment surface oriented in facing relationship with respect to the first treatment surface and angularly movable about a turn shaft parallel to the first treatment surface and into abutment against the first treatment surface wherein
when the first treatment surface and the second treatment surface are held in abutment against one another,
the first insulative surface abuts against the second electrode and the second insulative surface, and
the second insulative surface abuts against the first electrode and the first insulative surface,
thereby preventing a short circuit between the first electrode and the second electrode.

2. The treatment tool of claim 1, wherein
the second electrode is made up of a plurality of separate electrode members having the same pole and kept at the same electrical potential or voltage.

3. The treatment tool of claim 1, wherein
the first insulative surface has an electrode abutment surface for abutting against the second electrode and an insulation abutment surface for abutting the second insulative surface.

4. The treatment tool of claim 1, wherein
either the first treatment surface or the second treatment surface is formed as a projected surface.

5. The treatment tool of claim 1, further comprising:
a heating means configured to be used to treat a biotissue during the treatment.

6. The treatment tool of claim 5, wherein the heating means is a heater.

7. The treatment tool of claim 5, wherein the heating means is an ultrasonic energy to generate heat for the treatment.

8. The treatment tool of claim 1, wherein
the first electrode includes a first surface;
the second electrode includes a second surface;
a length of the first insulative surface along the widthwise directions is larger than a length of the second surface along the widthwise directions; and
a length of the second insulative surface along the widthwise directions is larger than a length of the first surface along the widthwise directions.

9. The treatment tool of claim 1, wherein
each of the first insulative surface and the second insulative surface is made of fluororesin.

10. The treatment tool of claim 1, wherein
the second insulative surface is defined by a pad that is electrically insulative.

11. The treatment tool of claim 1, wherein
the first electrode includes a first surface;
the second electrode includes a second surface;
the first treatment surface is a flat surface by the first insulative surface and the first surface; and
the second treatment surface is a flat surface by the second insulative surface and the second surface.

12. The treatment tool of claim 1, wherein
the first electrode includes a first surface;
the second electrode includes a second surface; and
at least one of a boundary between the first insulative surface and the first surface on the first treatment surface and a boundary between the second insulative surface and the second surface on the second treatment surface is formed as a flush surface.

13. The treatment tool of claim 1, wherein
the first treatment surface extends along the longitudinal axis;
the first treatment surface and the second treatment surface have slits defined respectively therein that extend along the longitudinal axis; and
a cutter is capable of being brought into and out of the slits along the longitudinal axis.

14. A treatment system comprising:
an energy source apparatus; and
an elongated treatment tool configured to be attached to the energy source apparatus to receive electrical energy, the elongated treatment tool having a treatment portion disposed on a longitudinal axis thereof,
the treatment portion including:
a first treatment surface having a first electrode and a first insulative surface formed therein, the first electrode extends along the longitudinal axis and being disposed at a center in widthwise directions perpendicular to the longitudinal axis and the first insulative surface disposed outwardly with respect to the first electrode in the widthwise directions; and
a second treatment surface having a second insulative surface and a second electrode formed therein, the second insulative surface extends along the longitudinal axis and being disposed at the center in the widthwise directions perpendicular to the longitudinal axis and the second electrode disposed outwardly with respect to the second insulative surface in the widthwise directions, the second treatment surface oriented in facing relationship with respect to the first treatment surface and angularly movable about a turn shaft parallel to the first treatment surface and into abutment against the first treatment surface wherein
when the first treatment surface and the second treatment surface are held in abutment against one another,
the first insulative surface abuts against the second electrode and the second insulative surface, and
the second insulative surface abuts against the first electrode and the first insulative surface,
thereby preventing a short circuit between the first electrode and the second electrode.

15. The treatment system of claim 14, wherein
the second electrode is defined by a plurality of separate electrode members having the same pole and the same electrical potential or voltage.

16. The treatment system of claim 14, wherein
the first insulative surface has an electrode abutment surface for abutting against the second electrode and an insulation abutment surface for abutting the second insulative surface.

17. The treatment system of claim 14, wherein
One of the first treatment surface or the second treatment surface is formed as a projected surface.

18. A treatment system comprising:
an energy source apparatus; and
an elongated treatment tool configured to be attached to the energy source apparatus to receive electrical energy, the elongated treatment tool includes a main body, a shaft, and a treatment portion all of which being attached to one another and being disposed on a longitudinal axis thereof
the treatment portion including:
a first treatment surface having a first electrode and a first insulative surface formed therein, the first electrode extends along the longitudinal axis and being disposed at a center in widthwise directions perpendicular to the longitudinal axis and the first insulative surface disposed outwardly with respect to the first electrode in the widthwise directions; and
a second treatment surface having a second insulative surface and a second electrode formed therein, the second insulative surface extends along the longitudinal axis and being disposed at the center in the widthwise directions perpendicular to the longitudinal axis and the second electrode disposed outwardly with respect to the second insulative surface in the widthwise directions, the second treatment surface oriented in facing relationship with respect to the first treatment surface and angularly movable about a turn shaft parallel to the first treatment surface and into abutment against the first treatment surface wherein
when the first treatment surface and the second treatment surface are held in abutment against one another,
the first insulative surface abuts against the second electrode and the second insulative surface, and
the second insulative surface abuts against the first electrode and the first insulative surface,
thereby preventing a short circuit between the first electrode and the second electrode while the electrode and the insulative surfaces are in flat contact with no gap.
